(19) United States
(12) Patent Application Publication
(10) Pub. No.: US 2025/0222176 A1
(43) Pub. Date: Jul. 10, 2025

(54) DEVICES AND METHODS FOR REDUCING OR PREVENTING CENTRAL VENOUS STENOSIS

(71) Applicant: CERULEAN SCIENTIFIC INC., Durham, NC (US)

(72) Inventors: Todd MCFARLAND, Durham, NC (US); Lora ALLEMEIER, Durham, NC (US)

(21) Appl. No.: 19/014,828

(22) Filed: Jan. 9, 2025

Related U.S. Application Data
(60) Provisional application No. 63/619,277, filed on Jan. 9, 2024.

(51) Int. Cl. A61L 29/06; A61L 29/08; A61M 25/00

(52) U.S. Cl. CPC A61L 29/06; A61L 29/08; A61M 25/0045; A61L 2400/10

(57) ABSTRACT

Disclosed herein are methods of reducing or preventing the complications associated with the percutaneous placement of central venous catheters within a patient, including methods of reducing or preventing central venous stenosis. These methods can involve coating at least a portion of the exterior of the central venous catheter with a coating that reduces friction between the exterior surface of the catheter body and an endothelial wall of the vein. Upon placement the catheter within the vein of the patient, the coating can reduce friction between the catheter body and the walls of the blood vessel, thereby reducing irritation and/or injury of the blood vessel.

### CROSS-REFERENCE TO RELATED APPLICATION

This application claims priority to, and the benefit of, U.S. Provisional Application Ser. No. 63/619,277, filed Jan. 9, 2024, the disclosure of which is expressly incorporated herein by reference in its entirety.

### BACKGROUND

The central venous catheter is a medical device which can allow blood sampling, blood pressure monitoring, and the administration of fluid, blood and medication to a patient. A central venous catheter is percutaneously inserted, via the Seldinger technique, such that the distal catheter tip resides within a large caliber vein, and the proximal portion of the catheter lies external to the patient's body. Typically, a central venous catheter is inserted into an internal jugular vein or subclavian vein with its distal tip residing within the superior vena cava. After the placement of a central venous, a chest x-ray is obtained to identify that the distal tip of the catheter is appropriately positioned in the region of the superior vena cava prior to being used. The central venous catheter is commonly used, particularly in hospital intensive care units, due to the need for reliable venous access in the medical management of acutely ill patients.

The central venous catheter has become a staple item in helping medical personnel care for patients, but it is also associated with numerous immediate and delayed complications. Immediate complications can arise at the time of the catheter's insertion and can include, but are not limited to, hematomas or bleeding, inadvertent cannulation of an artery, pneumothorax or cardiac arrhythmias. With appropriate technique and ultrasound guidance many of the immediate complications can be avoided. The delayed complications associated with central venous catheters can present significant obstacles in managing a patient's healthcare.

When a central venous catheter is inserted into the lumen of a vein, proteins begin to deposit along the surface of the catheter creating a catheter related sheath (CRS). These sheaths can be asymptomatic, but can also result in numerous delayed complications:

When the CRS covers the catheter's opening, it can effectively occlude the central venous catheter.

If the CRS wraps around the entire length of the indwelling central venous catheter it can result in medical extravasation into the adjacent soft tissues as the medication tracks retrograde in between the catheter opening and sheath to the entry site of the catheter into the vein.

The CRS enhances central venous catheter infections and persistent bacteremia.

The CRS can lead to a stenosis or occlusion of the vein in which the catheter resides. A central venous stenosis poses significant problem to patients, particularly chronic end-stage renal dialysis patients with upper extremity arterial venous fistulas or grafts, who need adequate venous outflow to maintain patency of their fistulas or grafts.

A significant length of the central venous catheter lies within the lumen of a vein which provides a large surface area for CRS development and its subsequent complications. The distal tip of a central venous catheter is mobile within the lumen of a vein and constantly moves due to blood flow, respiration and cardiac motion. This constant motion results in repeated micro-trauma to the adjacent vein wall, which can result in thrombus formation along the catheter tip or adjacent vein wall. This thrombus can result in catheter malfunction, by obstructing the catheter lumen, or stenosis of the vein lumen.

The complications associated with central venous catheters pose significant issues with regards to managing the healthcare of acutely ill patients. Accordingly, provided catheters and methods that reduce or prevent these complications, including central venous stenosis, are needed.

### SUMMARY

Provided herein are methods of reducing or preventing the complications associated with the percutaneous placement of central venous catheters (e.g., hemodialysis catheters) within a patient, including methods of reducing or preventing central venous stenosis. These methods can involve coating at least a portion of the exterior of the central venous catheter with a coating that reduces friction between the exterior surface of the catheter body and an endothelial wall of the vein. Upon placement the catheter within the vein of the patient, the coating can reduce friction between the catheter body and the walls of the blood vessel, thereby reducing irritation and/or injury of the blood vessel (which may drive stenosis).

Accordingly, provided herein are methods of reducing or preventing central venous stenosis in a patient having a percutaneously inserted central venous catheter present within a vein. These methods can comprise providing central venous catheter comprising: a catheter body having an exterior surface and comprising a proximal end, a distal end, and a mid-section; and a main lumen having an interior lumen wall, the main lumen running along the length of said catheter, thereby defining a main axis, and terminating at a distal lumen orifice, wherein such distal lumen orifice is arranged to provide liquid communication from said main lumen to the vein when the central venous catheter is percutaneous inserted within the vein; disposing a lubricant coating on at least a portion of the exterior surface of the catheter body disposed with in the vein when the central venous catheter is percutaneous inserted within the vein; and percutaneously inserting the central venous catheter within the vein of the patient. The lubricant coating can reduce friction between the exterior surface of the catheter body and an endothelial wall of the vein.

In some embodiments, the lubricant coating can exhibit a coefficient of friction of 0.5 or less with the endothelial wall of the vein.

Also provided herein are central venous catheters (e.g., hemodialysis catheters) comprising: a catheter body having an exterior surface and comprising a proximal end, a distal end, and a mid-section; a main lumen having an interior lumen wall, the main lumen running along the length of said catheter, thereby defining a main axis, and terminating at a distal lumen orifice, wherein such distal lumen orifice is arranged to provide liquid communication from said main lumen to a vein of a patient when the central venous catheter is percutaneous inserted within the vein; and a lubricant coating on at least a portion of the exterior surface of the catheter body disposed with in the vein of a patient when the central venous catheter is percutaneous inserted within the vein; The lubricant coating can reduce friction between the exterior surface of the catheter body and an endothelial wall of the vein.

In some embodiments, the lubricant coating can exhibit a coefficient of friction of 0.5 or less with the endothelial wall of the vein.

Further aspects of suitable lubricant coatings are described below and recited in the following claims.

### BRIEF DESCRIPTION OF DRAWINGS

FIG. 1 shows an exemplary illustration of a central line catheter.

FIG. 2 shows an exemplary illustration of the placement of a central line catheter.

FIGS. 3A-3D show results of a 90 day sheep study of coated catheter (TLP) vs uncoated catheter. FIG. 3A shows control/palindrome catheter after 90 days in sheep carotid vein; FIG. 3B shows treated/TLP catheter in same sheep after 90 days in separate carotid vein; FIG. 3C shows treated/TLP being removed from vein (no stenosis in vein); and FIG. 3D shows an illustration of placement of both control and treated catheters.

### DETAILED DESCRIPTION

Provided herein are methods of reducing or preventing the complications associated with the percutaneous placement of central venous catheters (e.g., hemodialysis catheters) within a patient, including methods of reducing or preventing central venous stenosis. These methods can involve coating at least a portion of the exterior of the central venous catheter with a coating that reduces friction between the exterior surface of the catheter body and an endothelial wall of the vein. Upon placement the catheter within the vein of the patient, the coating can reduce friction between the catheter body and the walls of the blood vessel, thereby reducing irritation and/or injury of the blood vessel (which may drive stenosis).

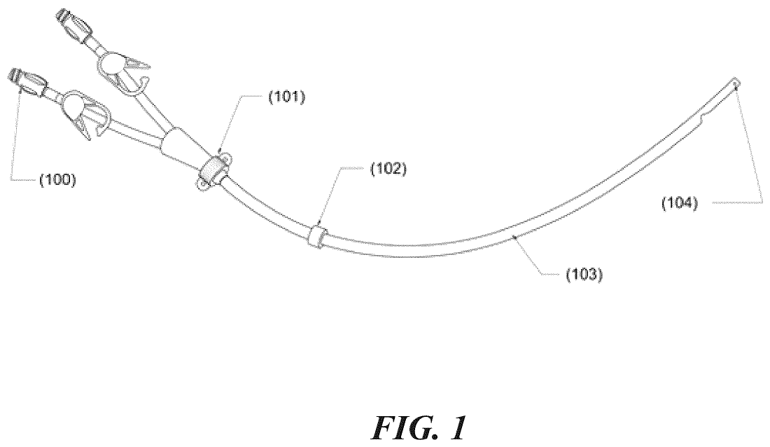

Referring now to FIG. 1, the central venous catheter includes polymer luers 100, suture wings 101, polymer cuff 102, catheter body 103, catheter holes 104.

In some aspects, the central venous catheter includes a catheter body having an exterior surface and including a proximal end, a distal end, and a mid-section; a main lumen having an interior lumen wall, the main lumen running along the length of said catheter, thereby defining a main axis, and terminating at a distal lumen orifice, wherein such distal lumen orifice is arranged to provide liquid communication from said main lumen to a vein of a patient when the central venous catheter is percutaneous inserted within the vein; and a lubricant coating on at least a portion of the exterior surface of the catheter body disposed with in the vein of a patient when the central venous catheter is percutaneous inserted within the vein; wherein the lubricant coating reduces friction between the exterior surface of the catheter body and an endothelial wall of the vein.

In some aspects, the lubricant coating exhibits a coefficient of friction of 0.5 or less with the endothelial wall of the vein.

In some aspects, the lubricant coating includes a roughened substrate and a lubricating fluid layer immiscible with a biological material, the lubricating fluid layer adhering to and preferentially wetting the roughened substrate to form a slippery surface over the roughened substrate.

In some aspects, the roughened substrate includes a metal. In some aspects, the metal includes a shape memory metal, such as nitinol.

In some aspects, the roughened substrate includes a polymer. In some aspects, the roughened substrate includes a porous polymer. In some aspects, the porous polymer includes PTFE, ePTFE, silicon, polyurethane, or combinations thereof.

In some aspects, the roughened substrate includes roughened features of 50 nm to 1 mm.

In some aspects, the lubricating fluid includes a fluorocarbon, such as a perfluorocarbon. In some aspects, the fluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1H-Perfluoropentane, HFA 134aTM, HFA227eaTM, methyl perfluorobutylether, methyl perfluoropropyl ether (3M Novec 7000™), 2,2,2-trifluoroethanol, silicone solutions, and combinations thereof.

In some aspects, the lubricating fluid further includes carbohydrates, proteins, fats, vitamins, minerals, electrolytes, and combinations thereof.

In some aspects, the lubricating fluid is insoluble and immiscible in water.

In some aspects, the catheter further includes an anti-biofouling coating on the interior lumen wall. In some aspects, the anti-biofouling coating includes a roughened substrate and a lubricating fluid layer immiscible with a biological material, the lubricating fluid layer adhering to and preferentially wetting the roughened substrate to form a slippery surface over the roughened substrate.

Additional description of lubricant coating can be found in U.S. Pat. No. 9,932,484 (Aizenberg, J. et al), which is expressly incorporated herein by reference.

In some aspects, the central venous catheter includes a hemodialysis catheter.

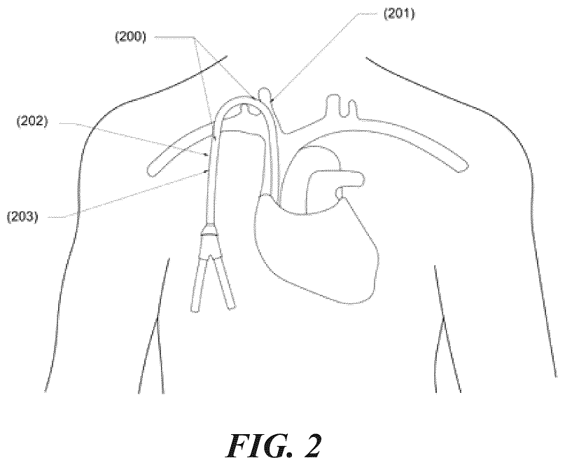

Referring now to FIG. 2, the methods disclosed herein relate to the placement of a central venous catheter including vascular graft 200, artery 201, vein 202, access needles 203, 204 for hemodialysis, location 205,206 of hemodialysis machine (when applicable).

In some aspects, an exemplary method of reducing or preventing central venous stenosis in a patient having a percutaneously inserted central venous catheter present within a vein is disclosed. The method includes providing central venous catheter including: a catheter body having an exterior surface and including a proximal end, a distal end, and a mid-section; and a main lumen having an interior lumen wall, the main lumen running along a length of said catheter, thereby defining a main axis, and terminating at a distal lumen orifice, wherein such distal lumen orifice is arranged to provide liquid communication from said main lumen to the vein when the central venous catheter is percutaneous inserted within the vein; disposing a lubricant coating on at least a portion of the exterior surface of the catheter body disposed with in the vein when the central venous catheter is percutaneous inserted within the vein; and percutaneously inserting the central venous catheter within the vein of the patient; wherein the lubricant coating reduces friction between the exterior surface of the catheter body and an endothelial wall of the vein.

In some aspects of the exemplary method, the lubricant coating exhibits a coefficient of friction of 0.5 or less with the endothelial wall of the vein.

In some aspects of the exemplary method, the lubricant coating includes a roughened substrate and a lubricating fluid layer immiscible with a biological material, the lubricating fluid layer adhering to and preferentially wetting the roughened substrate to form a slippery surface over the roughened substrate. In some aspects, the roughened substrate includes a polymer. In some aspects, the roughened substrate includes a porous polymer. In some aspects, the porous polymer includes PTFE, ePTFE, silicon, polyurethane, fluorinated ethylene propylene, or combinations thereof. In some aspects, the roughened substrate includes roughened features of 50 nm to 1 mm.

In some aspects of the exemplary method, the lubricating fluid includes a fluorocarbon. In some aspects, the fluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1HPerfluoropentane, HFA 134aTM, HFA227caTM, methyl perfluorobutylether, methyl perfluoropropyl ether (3M Novec 7000™), 2,2,2-trifluoroethanol, silicone solutions, and combinations thereof. In some aspects, the lubricating fluid further includes carbohydrates, proteins, fats, vitamins, minerals, electrolytes, and combinations thereof. In some aspects, the lubricating fluid is insoluble and immiscible in water.

In some aspects of the exemplary method, the catheter interior lumen wall further includes an anti-biofouling coating. In some aspects, the anti-biofouling coating includes a roughened substrate and a lubricating fluid layer immiscible with a biological material, the lubricating fluid layer adhering to and preferentially wetting the roughened substrate to form a slippery surface over the roughened substrate. In some aspects, the roughened substrate includes a polymer. In some aspects, the roughened substrate includes a porous polymer. In some aspects, the porous polymer includes PTFE, ePTFE, silicon, polyurethane, or combinations thereof. In some aspects, the roughened substrate includes roughened features of 50 nm to 1 mm. In some aspects, the lubricating fluid includes a fluorocarbon, such as a perfluorocarbon. In some aspects, the fluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1HPerfluoropentane, HFA 134aTM, HFA227caTM, methyl perfluorobutylether, methyl perfluoropropyl ether (3M Novec 7000™), 2,2,2-trifluoroethanol, silicone solutions, and combinations thereof. In some aspects, the lubricating fluid further includes carbohydrates, proteins, fats, vitamins, minerals, electrolytes, and combinations thereof.

### Lubricant Coatings

In some embodiments, the lubricant coating can include a self-healing layer of lubricating fluid on a surface of the medical device. The surface can be formed in whole or in part from any suitable material for fabrication of a medical device, such as a metal (e.g., a shape memory polymer) or a polymer. The surface can have a roughened texture that interacts with and attracts the lubricating fluid to form a lubricating fluid layer. The roughened surface may have features that are on the scale of 50 nm to 100 microns, 50 nm to 500 microns, 500 nm to 1 mm, or 500 microns to 1 mm. In certain examples, roughened substrate can comprise a porous polymer, such as PTFE, ePTFE, silicon, polyurethane, or combinations thereof. The roughened substrate can include porous features of from 50 nm to 1 mm.

In some examples, the lubricating fluid includes a perfluorocarbon, wherein the perfluorocarbon includes perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4Hperfluorobutane, 1H-Perfluoropentane, HFA 134aTM, HFA227caTM, methyl perfluorobutylether, methyl perfluoropropyl ether (3M Novec 7000™), 2,2,2-trifluoroethanol, silicone solutions, and combinations thereof. The lubricating fluid may further include carbohydrates, proteins, fats, vitamins, minerals, electrolytes, and combinations thereof.

Examples of suitable lubricating coatings are described, for example, in U.S. Pat. No. 10,982,100, which is incorporated by reference in its entirety. Further examples of suitable substrates and lubricating fluids are described below.

### Substrates

In one embodiment, the substrate is a low-surface energy porous solid. In the disclosed embodiments, the substrate is preferentially wetted by the lubricating fluid rather than by the fluid to be repelled. It can have a roughened or smooth surface. As used herein, the term “roughened surface” is a substrate that includes both the surface of a three-dimensionally porous material as well as solid surface having certain topographies, whether they have regular, quasiregular, or random patterns. In some embodiments, the substrate is roughened by incorporation of microtextures. In other embodiments, the substrate is roughened by incorporation of nanotextures. Physically, the large surface area provided by micro/nanoscale roughness not only facilitates complete wetting by the lubricating fluid but also strengthens the adhesion of lubricating fluid (Liquid B) within the porous solid.

Lubricant coatings can have properties that are insensitive to the precise geometry of the underlying substrate. Therefore, the geometry of the substrate can be any shape, form, or configuration to suit various-shaped materials and devices. In certain embodiments, the porous surface can be manufactured over any suitable materials and geometries, such as medical devices, inside of pipes (e.g., metallic or metallized pipes), optical windows, biological sensor windows, medical tubing, hollow metallic structures, patterned electrodes, meshes, wires, porous conductive surfaces, and the like that come into contact with biological materials.

Non-limiting examples of shapes, forms, and configurations the coating can take include generally spherical (e.g., bead, magnetic particles, and the like), tubular (e.g., for a cannula, connector, catheter, needle, capillary tube, tubing, or syringe), planar (e.g., for application to a microscope slide, plate, film, or laboratory work surface), or arbitrarily shaped (e.g., well, well plate, Petri dish, tile, jar, flask, beaker, vial, test tube, column, container, cuvette, bottle, drum, vat, or tank). For example, the coating can be applied to spherical surfaces, such as magnetic particles that can be actuated inside the body for drug delivery.

The roughened surface can include a variety of features (e.g., raised structure, protrusions, or pores) and can be randomly oriented or ordered on a surface. A range of surface structures with different feature sizes and porosities can be used. Feature sizes can be in the range of hundreds of nanometers to microns (e.g., 100 to 1000 nm), and have aspect ratios from about 1:1 to 10:1. Porous nano-fibrous structures can be generated in situ on the inner surfaces of metallic microfluidic devices using electrochemical deposition using techniques known in the art (Aizenberg, J., Kim, P. Hierarchical Structured Surfaces Resistant to Wetting by Liquids. U.S. Provisional Patent Application No. 61/353,505, filed on Jul. 19, 2010; Kim, P., Epstein, A. K., Khan, M., Zarzar, L. D., Lipomi, D. J., Whitesides, G. M., Aizenberg, J. Structural Transformation by Electrodeposition on Patterned Substrates (STEPS): A New Versatile Nanofabrication Method”, Nano Letters, in press (2011)).

In certain embodiments, the surface has a large surface area that is readily wetted by the lubricating fluid and which entrains lubricating fluid and retains it on the substrate surface. In certain embodiments, the substrate surface is a hierarchical surface containing surface features on multiple dimension scales. By way of example, the surface can have a first topological feature having dimensions on the microscale and a second topological feature on the nanoscale. The first topological feature supports the second smaller topological feature. The second topological features are referred to as “primary structures” as they are meant to denote the smallest feature sizes of the hierarchical structure. The primary structures can include structures, such as nanofibers, nanodots, and the like. Such nanoscale “primary structures” can have at least one kind of feature sizes that are a few to tens or hundreds of nanometers in size, such as less than 5 nm to 200 nm. For example, nanofibers having diameters of approximately 5, 10, 25, 50, or even 100 nm. In such cases, when “primary structures” having feature sizes of about 100 nm diameter is utilized, “secondary structures” having feature sizes that are larger than 100 nm, such as 150 nm, 300 nm, 500 nm, or 1000 nm, and larger. Additional higher order structures, such as “tertiary structures” and the like, each has larger feature sizes than the lower order structures are contemplated.

Particularly, hierarchical structures having nanofibers as the primary structures may provide a high degree of three-dimensional porosity that may be well-suited for use as porous surfaces described herein. A detailed discussion of hierarchical surfaces suitable for use with a liquid to be repelled is found in International Application No. PCT/US11/44553 entitled “Hierarchically structures surfaces to control wetting by liquids,” filed on Jul. 19, 2011, which is incorporated in their entirety by reference.

In certain embodiments, the roughened surface may have a periodic array of surface protrusions (e.g., posts, peaks, etc.) or any random patterns or roughness. In some embodiments, the size of the features producing a roughened surface range from 10 nm to 100 μm with geometries ranging from regular posts/open-grid structures to randomly oriented spiky structures. In some embodiments, the widths of the raised structures are constant along their heights. In some embodiments, the widths of the raised structures increase as they approach the basal surface from the distal ends. The raised structures can be raised posts of a variety of cross-sections, including, but not limited to, circles, ellipses, or polygons (such as triangles, squares, pentagons, hexagons, octagons, and the like), forming cylindrical, pyramidal, conical or prismatic columns. Although the exemplary substrates described above illustrate raised posts having uniform shape and size, the shape, orientation and/or size of raised posts on a given substrate can vary.

Polymeric materials, such as open porosity PTFE (ePTFE) membranes, can be pressed or molded to take on a variety of shapes.

In certain embodiments, the roughened surface has a roughness factor, R, greater than 1, where the roughness factor is defined as the ratio between the real surface area and the projected surface area. For complete wetting of lubricating fluid to occur, it is desirable to have the roughness factor of the roughened surface to be greater or equal to that defined by the Wenzel relationship (i.e., R≥1/cos θ, where θ is the contact angle of lubricating fluid on a flat solid surface). For example, if lubricating fluid has a contact angle of 50° on a flat surface of a specific material, it is desirable for the corresponding roughened surface to have a roughness factor greater than ˜1.5.

The roughened surface material can be selected to be chemically inert to the lubricating fluid and to have good wetting properties with respect to lubricating fluid. In addition, the roughened surface topographies can be varied over a range of geometries and size scale to provide the desired interaction, e.g., wettability, with lubricating fluid.

In certain embodiments, the micro/nanoscale topographies beneath the lubricating fluid enhance the liquid-wicking property and the adherence of lubricating fluid to the roughened surface. As a result, the lubricating fluid can uniformly coat the roughened surface and get entrapped inside at any tilting angles.

Non-limiting examples of porous materials include solid substrates having holes (e.g., high aspect ratio holes, cylinders, columns, etc.), three-dimensionally interconnected network of holes and one or more materials (e.g., 3-D ordered colloidal assemblies, block copolymers, etc.), and random array of fibrous materials (e.g., filter paper, fabrics, electrospun films).

Non-limiting examples of porous or rough surface structures that can be used include polymers (e.g., polysulfone, PDMS, and polypyrrole) and hydrophobic porous (e.g., Teflon) materials. For example, the roughened surface can be manufactured from polymers (e.g., epoxy, polycarbonate, polyester, nylon, etc.), metals, sapphire, glass, carbon in different forms (such as diamond, graphite, black carbon, etc.), ceramics (e.g., alumina), and the like. For example, fluoropolymers such as polytetrafluoroethylene (PTFE), polyvinylfluoride, polyvinylidene fluoride, fluorinated ethylene propylene, and the like can be used as substrates. Many porous materials are commercially available, or can be made by a number of well-established manufacturing techniques. For example, polytetrafluoroethylene (also known by the trade name “Teflon” and abbreviation “PTFE”) filter materials are commercially available. In some embodiments, the roughened surface is manufactured from a hemocompatible material, nonlimiting examples of which include silicon rubber and polysulfones. In certain embodiments, the roughened surface is manufactured from any suitable materials.

In certain embodiments, if the desired material and shape is not electrically conducting, the surfaces of such material and shapes can be rendered electrically conductive by applying a thin layer of conductive material, such as through vapor deposition techniques, sputtering, metallization techniques, and the like. Moreover, the porous surface can be readily formed on large surface area materials that are commercially important. When necessary, surface functionalization can be carried out to modify the solid surfaces so that the lubricating layer preferentially wets the roughened surface as compared to Liquid A.

The raised structures can be produced by any known method for fabricating raised structures onto substrates. Non-limiting examples include molding into the device structure, conventional photolithography, projection lithography, e-beam writing or lithography, depositing nanowire arrays, growing nanostructures on the surface of a substrate, soft lithography, replica molding, solution deposition, solution polymerization, electropolymerization, electrospinning, electroplating, vapor deposition, contact printing, etching, bead blasting, sand blasting, transfer patterning, microimprinting, self-assembly, and the like.

In certain embodiments, the roughened surface can be made, for example, by replica molding procedure described in B. Pokroy, A. K. Epstein, M. C. M. Persson-Gulda, J. Aizenberg, Adv. Mater. 21, 463 (2009), the contents of which is incorporated by reference herein in its entirety. Patterned surfaces can also be obtained as replicas (e.g., epoxy replicas) by a soft lithographic method (see, e.g., J. Aizenberg and B. Pokroy, PCT/US2009/048880, the contents of which is incorporated by reference herein in its entirety). Polymer films with patterned surfaces can be fabricated by means known in the art (e.g., roll-to-roll imprinting or embossing).

By way of non-limiting example, negative replicas of pre-generated patterns can be made from polydimethylsiloxane, PDMS (e.g., Dow-Sylgard 184) by pouring mixture of prepolymer and curing agent (e.g., 10:1 ratio) on the patterns followed by thermal curing in an oven. After cooling, the negative PDMS mold can be peeled off and used for fabricating the final replica by pouring the desired material (e.g. UV-curable epoxy resin) into the negative mold. After solidifying the material, the negative mold can be peeled off, leaving the replica of the original pattern. Then, the surface of the replica can be chemically functionalized with low surface energy coating such as (tridecafluoro-1,1,2,2-tetrahydrooctyl)-trichlorosilane.

For example, a silicon substrate having a post array can be fabricated by photolithography using the Bosch reactive ion etching method (as described in Plasma Etching: Fundamentals and Applications, M. Sugawara, et al., Oxford University Press, (1998), ISBN-10: 019856287X, the contents of which is incorporated by reference herein in its entirety). Arrays of hydrophobic raised surface structures can be made at the micrometer scale using micromolding techniques. For example, rough surface structures can be arrays of hydrophobic raised surface structures at the micrometer scale, such as posts and intersecting walls patterned in polymers such as epoxy.

In certain embodiments, the roughened surface may be the surface of a three dimensionally porous material. The porous material can be any suitable porous network having a sufficient thickness to stabilize lubricating fluid, such as a thickness from about 5 μm to about 1 mm. Moreover, the porous material can have any suitable pore sizes to stabilize the lubricating fluid, such as from about 10 nm to about 100 μm.

In another embodiment, porous alumina is manufactured by the process of anodization, where an aluminum substrate is electrochemically oxidized under constant electrical potential. The pore size, inter-pore spacing, and aspect ratio of the pores can be tuned by adjusting the operating parameters of the electrochemical oxidation process. Such a process generates porous through-holes into the substrate, where the size of the porous holes are on the order of 50 nm with aspect ratio larger than 10000 (see, Lee et al., Nature Mater. 5, 741-47, 2006, the contents of which is incorporated by reference herein in its entirety).

In some embodiments, mechanical or (electro) chemical methods can be used to roughen metal surfaces. Roughening and non-wetting materials can be spray coated directly onto metal surfaces. Boehmite (γ-AlO(OH)) formation on aluminum surface by boiling in water can also be used to roughen metallic surfaces such as aluminum. Rotary jet spinning of hydrophobic polymer nanofibers and layered deposition of an appropriate primer can also be used to roughen substrates.

In yet another embodiment, long range ordered porous structures of silica can be produced by evaporative co-assembly method of sacrificial polymeric colloidal particles together with a hydrolyzed silicate sol-gel precursor solution. Such a method generates a crack-free porous surface on the order of centimeters or larger, with pore sizes of about 100 nm to about 1000 nm and porosity of about 75%. (See, Hatton, et al., Proc. Natl. Acad. Sci. 107, 1035410359, 2010 and U.S. patent application Ser. No. 13/058,611, filed on Feb. 11, 2011, the contents of which is incorporated by reference herein in its entirety).

Polymer-based porous membranes (such as medical grade PTFE) can be made by mixing PTFE powders with lubricating fluid to form a paste. Then, the paste can be molded into the desired shape by methods such an extrusion molding. The molded PTFE membrane can then be heated up to less than its melting point to drive off the lubricants. Thereafter, a porous PTFE membrane can be formed (see U.S. Pat. No. 5,476,589, the content of which is incorporated by reference herein in its entirety).

In yet another embodiment, the porous material can be generated in-situ on a metal surface by an electrodeposition method, such as the STEP method (STEP=structural transformation by electrodeposition on patterned substrates, see, PCT Application No. PCT/US11/44553, filed on Jul. 19, 2011, and Kim, et al., Nano Lett., in press, (2011), the contents of which are incorporated by reference herein in their entirety. The electrodeposition condition can be controlled so that nanofibers of electrically conductive polymer can be formed over an electrically conductive surface. The electrodeposition conditions can further be controlled to provide a desired nanofiber diameter and spacing. In certain embodiments, the electrodeposition condition can be controlled to provide any other desirable morphology that can provide additional means to stabilize the lubricating layer.

The morphology of the conducting organic polymers can be controlled by varying the deposition conditions such as the concentration of monomer, the types of electrolytes and buffers, the deposition temperature and time, and the electrochemical conditions such as applied potential. For example, increasing the concentration of monomer in the electrochemical solution, the applied potential, and/or the temperature generally leads to a faster polymerization rate and many parasitic nucleation sites during growth resulting in a morphology that is similar to a cauliflower. In contrast, lower concentrations of monomer, lower applied potential, and lower temperatures can lead to nanofibrile growth with substantially uniform diameters. Further decrease in concentration of monomer or applied potential can lead to short rods of polymer nanofibers with low surface coverage. In another example, increasing the type of electrolytes and buffers to obtain a more acidic solution can lead to the formation of a cauliflower shape or overgrowth of polymers. In another example, the applied voltage can be cycled leading to different oxidation states of the deposited polymer layer which is often manifested as a color change (e.g., from dark blue to a green then to a pale yellow color with increasing applied voltage).

In yet another example, the applied voltage can be pulsed at a constant voltage to form polymers only on the tip of the underlying micropost structures, leading to a mushroom-like morphology. Accordingly, the morphology of conducting organic polymers can be finely controlled from nanometers to over micrometer scales, and surface coatings with precisely controlled morphology can be produced by simple modifications, which promise the customization of various surface properties by design and control of the morphology. In other embodiments, a roughened surface is further functionalized to improve wetting by lubricating fluid. Surface coating can be achieved by methods well known in the art, including plasma assisted chemical vapor deposition, chemical functionalization, solution deposition, and vapor deposition. For example, surfaces containing hydroxyl groups (i.e., —OH) can be functionalized with various commercially available fluorosilanes (e.g., tridecafluoro1,1,2,2-tetrahydrooctyl-trichlorosilane, heptadecafluoro-1,1,2,2-tetra-hydrodecyl trichlorosilane, etc.) to improve wetting by low surface tension fluids.

In certain embodiments, many materials having native oxides, such as silicon, glass, and alumina, can be activated to contain —OH functional groups using techniques such as plasma treatment. After activation, either vapor or solution deposition techniques can be used to attach silanes so that surfaces with low surface energy can be produced. For vapor deposition, the deposition can be carried out by exposing the surface to silane vapors. For solution deposition, the deposition can be carried out by immersing the surface in a silane solution, followed by rinsing and blow-drying after deposition. For layered deposition, layered deposition of a primer is followed by application of a mixture of sacrificial beads and Liquid B, which is dried and cured. The beads are removed to produce a contiguous porous Teflon-like surface.

In some other embodiments, where hydroxyl groups is absent on the surface, the surface can be functionalized by first coating it with thin films of metals, such as gold or platinum, and the thin metal films can be functionalized with various commercially available thiols of low surface energy (e.g., heptane thiol, perfluorodecanethiol, etc.). Similarly, vapor or solution deposition techniques can be carried out similar to that describe for silane deposition using, for example, alkane thiol solutions.

In another embodiment, the roughened, porous substrate can be generated by a spraying method, where emulsions consisting of micro/nanoparticles are sprayed onto a flat solid surface. These particles assemble into roughened solid layer upon solvent drying. Such a solid layer can then be infiltrated by lubricating fluid (which can also be applied by additional spraying). Nonlimiting examples of micro/nanoparticles that can be sprayed onto a flat solid surface to form roughened, porous material include titanium dioxide, silicon dioxide, nanodiamonds, metals such as silver, gold, platinum, copper, gold, palladium, zinc, and titanium, hydroxyapatite (HAp) nanoparticles.

In one or more embodiments, the roughened, porous substrate is generated by chemical or physical etching, which includes mechanical roughening such as bead blasting and sand blasting. Once the surface is roughened, it is functionalized with a liquid or vapor, and infiltrated with a lubricating liquid.

In other embodiments, the roughened, porous substrate is made by growing a nanostructured material on the surface. Non-limiting examples of these nanostructures include PPy nanofibers, carbon nanotubes, and the like. One the nanostructures are in place, the surface can be chemically functionalized by silanization and infiltrated with a lubricating liquid.

In certain embodiments, the roughened surface can be formed over or applied to a variety of planar or non-planar surface. For example, a porous membrane can be attached to the outer surface of a cylindrical solid core. It can also be attached to the inner surfaces, outer surfaces, or inner and outer surfaces of tubes and other irregularly shaped substrates.

In certain embodiments, the solid surface may be substantially flat. This situation may be applicable when the critical surface energy of the flat surface is higher than the surface tension of the functional lubricating fluid.

In certain embodiments, the roughened surface can have pores that are comparable or smaller than the material to be repelled. For example, pore sizes that are smaller than the size of protozoa (e.g., 10 μm), bacteria (e.g., 1 μm), viruses (e.g., 0.1 μm), and the like can be utilized.

### Lubricating Fluids (Liquid B)

Lubricating fluids can be selected to create a fluid surface that is intrinsically smooth, stable, and defect free. The lubricating fluid should infiltrate, wet, and stably adhere to the substrate. Moreover, it should be chemically inert with respect to the solid substrate and the fluid to be repelled. In certain embodiments, a lubricating fluid possesses the ability to form a substantially molecularly flat surface when provided over a roughened surface. In certain other embodiments, a lubricating fluid possesses the ability to form a substantially atomically flat surface when provided over a roughened surface. In one or more embodiments, the lubricant is substantially incompressible.

Further, the lubricating fluid can be capable of repelling immiscible fluids, and in particular biological fluids of any surface tension. For example, the enthalpy of mixing between the fluid to be repelled and lubricating fluids be may sufficiently high (e.g., water and oil) that they phase separate from each other when mixed together. In certain embodiments, the lubricating fluid can be selected such that the fluid to be repelled has a small or substantially no contact angle hysteresis. For example, contact angle hysteresis less than about 5°, 2.5°, 2°, or even less than 1° can be obtained. Low contact angle hysteresis encourages sliding at low tilt angles (e.g.,) <5°, further enhancing fluid repellant properties of the surface.

The effectiveness of a given lubricating fluid's ability to repel fluids can be confirmed by visualization techniques known in the art including fluorescence microscopy and scanning electron microscopy (SEM).

In one or more embodiments, lubricating fluid is inert with respect to the solid surface and biological fluid. Lubricating fluid flows readily into the recesses of the roughened surface and generally possesses the ability to form an ultra-smooth surface when provided over the roughened surface. The structures present on the roughened substrate can greatly enhance the wetting of the lubricating fluid on the surface, creating a uniformly-coated slippery functional layer over the topographies. The resulting ultra-smooth surface is capable of repelling fluids including, but not limited to biological fluids and particles in solution or suspension.

Lubricating fluid can be selected from a number of different fluids. These fluids can be selected based on their biocompatibility, low (or high) toxicity, anti-clotting performance, chemical stability under physiological conditions, and levels of leaching from the surfaces of the devices. For example, compounds that are approved for use in biomedical applications (e.g., blood substitutes, MRI contrast agents), such as perfluorinated hydrocarbons and organosilicone compounds (e.g. silicone elastomer) can be used as lubricating fluids. In one or more aspects, the lubricating fluid is a chemically-inert, high-density biocompatible fluid, non-limiting examples of which include tertiary perfluoroalkylamines (such as perfluorotri-n-pentylamine, FC-70, perfluorotri-n-butylamine FC-40, etc), perfluoroalkylsulfides and perfluoroalkylsulfoxides, perfluoroalkylethers, perfluorocycloethers (like FC-77) and perfluoropolyethers (such as KRYTOX family of lubricants by DuPont), perfluoroalkylphosphines and perfluoroalkylphosphineoxides, and combinations thereof are used. In addition, long-chain perfluorinated carboxylic acids (e.g., perfluorooctadecanoic acid and other homologues), fluorinated phosphonic acids, fluorinated silanes, and combinations thereof can be used as Liquid B. Perfluoroalkyls can be linear or branched.

In certain embodiments, the lubricating fluid has a high density. For example, the lubricating fluid has a density that is more than 1.0 g/cm³, 1.6 g/cm³, or even 1.9 g/cm³. In certain embodiments, the density of the lubricating fluid is greater than that of the biological fluid to enhance fluid repellency. High density fluids reduce the tendency of an impacting fluid to ‘sink’ below the surface of lubricating fluid and to become entrained therein. In certain embodiments, the density of Liquid A may be lower than that of the lubricating liquid. For example, density of Liquid A may be at least ˜1.5 times lower than that of the lubricating liquid.

In certain embodiments, the lubricating fluid has a low evaporation rate, such as less than 100 nm/s, less than 10 nm/s, or even less than 1-2 nm/s. The lubricating fluid should be applied in a thickness sufficient to cover the rough surface of the substrate and provide an ultra-smooth surface. Taking a typical thickness of the lubricating fluid to be about 10 μm and an evaporation rate of about 1-2 nm/s, SLIPS can remain highly fluid-repellant for a long period of time without any refilling mechanisms.

In certain embodiments, lubricating fluid has a low freezing temperature, such as less than −5° C., −25° C., or even less than −50° C. Having a low freezing temperature allows lubricating fluid to maintain its slippery behavior to repel a variety of liquids or solidified fluids, such as ice and the like and over a range of temperatures.

Experimentally, it is observed that an object can become highly mobile on the surface of the lubricating liquid when the kinematic viscosity of the lubricating liquid is less than 1 cm²/s. Since liquid viscosity is a function of temperature (i.e., liquid viscosity reduces with increasing temperature), choosing the appropriate lubricant that operates at the aforementioned viscosity (i.e., <1 cm²/s) at specific temperature range is desirable. Particularly, various different commercially available lubricating liquid can be found at the specified viscosity, such as perfluorinated oils (e.g., 3M™ Fluorinert™ and Dupont™ Krytox® oils), at temperatures ranging from less than −80° C. to greater than 260° C.

Lubricating fluid can be deposited in any desired thickness, provided the top surface of lubricating fluid forms an ultra-smooth surface and is retained and interacts with the underlying surface. If the liquid layer is too thick, the upper surface is ‘unbound’ from the underlying surface and will flow with a liquid from the coating surface. The liquid layer that interacts with and is retained by the underlying surface is referred to as the ‘characteristic thickness’ of the liquid layer. The characteristic thickness will vary depending on the underlying surface and the ambient conditions, e.g., temperature, pressure, etc. Film thicknesses substantially on the order of the surface roughness peak-to-valley distance provide good fluid-solid interaction between the substrate and lubricating fluid. When the solid substrate is tilted at a position normal to the horizontal plane, lubricating fluids with thicknesses below a characteristic length scale remain substantially adhered to the roughened surface, whereas fluid layers above the characteristic length can flow, creating flow lines (surface defects) and disrupting the flatness of the fluid surface. For example, non-limiting thicknesses for the lubrication fluid (as measured from the valleys of the roughened surface are on the order of 5-20 μm when the peak to valley height is ˜5 μm.

In certain embodiments, lubricating fluid can be applied by pupating drops of the fluid onto the roughened surface, or by dipping the roughened surface into a reservoir carrying lubricating fluid. In some embodiments, lubricating fluid can be sprayed, cast, or drawn onto the roughened surface. The lubricating liquid can infiltrate the roughened surface by capillary action, which can wet the roughened surface and form a film on top of it. Lubricating fluid and the roughened surface can be both generated by a double-spraying process, where emulsions consisting of nano/microparticles are first sprayed onto a flat solid surface to form a substantially roughened solid layer, and then lubricating fluid can be sprayed onto this freshly formed layer for further infiltration. In addition, lubricating fluid may infiltrate into the pores of the roughened surface by capillary action and form an ultra-smooth film on top of the roughened surface. In certain embodiments, when sufficient quantity of the lubricating fluid is provided, the lubricating fluid may wet the entire roughened surface structure and form an ultra-smooth film over the underlying roughened surface.

### EXAMPLE

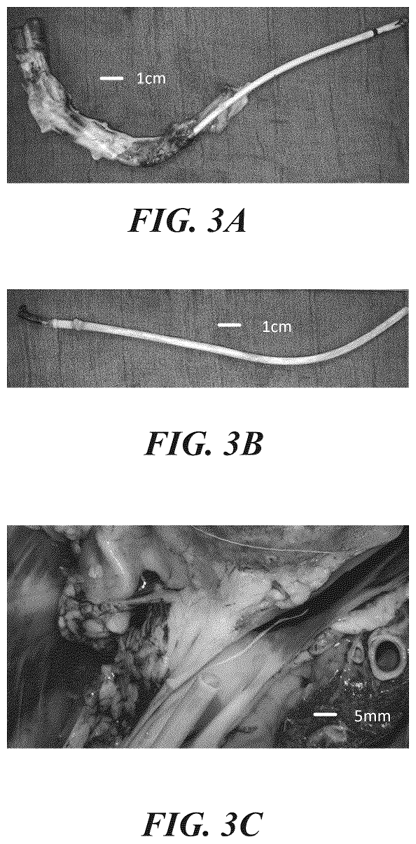

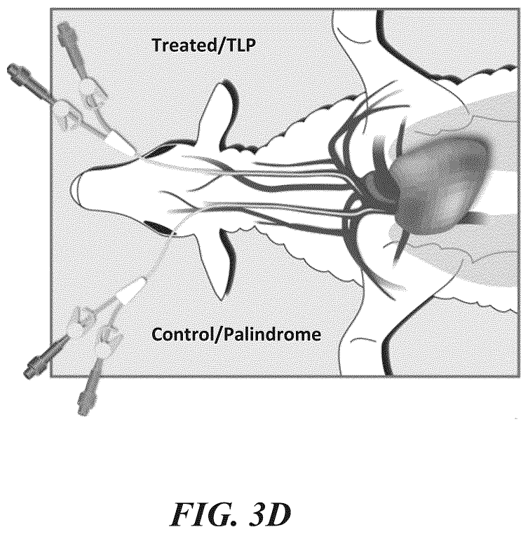

One coated (ePTFE coated with perfluorodecalin, termed TLP) hemodialysis catheter and one Medtronic Palindrome® polyurethane market leading control catheter were implanted into the right and left jugular veins of a domestic sheep for direct comparison (FIGS. 3A-3D). Both catheters were locked with sterile heparinized saline (100 U/mL) locking solution before implantation. The TLP HDC was flushed with liquid perfluorocarbon, and the control catheter was flushed with heparinized saline after implantation. Both catheters, under fluoroscopic guidance, were extended distally and both catheter tips were placed in the superior vena cava (SVC) without touching each other. Both catheter hubs were coiled inside the adjacent subcutaneous pocket, where it was sutured to the muscle layer using non-absorbable polypropylene suture. After 90 days of implantation, the sheep was euthanized following an IACUC-approved protocol. Both TLP and Palindrome® HDC were isolated with adjacent tissues and the surrounding tissue was dissected to expose the fibrous sheath. Fibrin sheath formation was calculated by measuring each area with a calibrated caliper. While Medtronic Palindrome® control showed 62% fibrin sheath formation, TLP HDC showed only 5.7% fibrin sheath. Catheter tips were inspected after opening both veins. In addition, the Palindrome® control tip and side holes were completely occluded with thrombus and tissue ingrowth, but the TLP HDC tip was completely free from thrombus. Catheter hubs from TLP-HDC were sonicated in PBS to dislodge any bacterial biofilm if formed. Subsequent microbiological evaluation reported zero CFU after 72 hours of incubation of three serial dilutions (10⁻¹, 10⁻², and 10⁻³). The TLP-coated HDC, demonstrated decreased thrombogenicity and pathogen colonization (>99%) on TLP-coated catheters vs. standard TPU catheters, and showed that coating effectiveness was maintained for 90 days under physiological flow conditions. No venous stenosis was noted in the coated catheter (ePTFE with liquid coating). This 90-day in vivo test demonstrates that an ePTFE vascular implant resists all thrombus formation, cellular adhesion, biofilm formation, and stenosis for at least 90 days.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The expressions “ambient temperature” and “room temperature” as used herein are understood in the art and refer generally to a temperature from about 20° C. to about 35° C.

As used herein, the term “composition” is intended to encompass a product comprising the specified ingredients in the specified amounts, as well as any product which results, directly or indirectly, from a combination of the specified ingredients in the specified amounts. References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such ratio regardless of whether additional components are contained in the mixture. A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example embodiments.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then about 1% by weight, e.g., less than about 0.5% by weight, less than about 0.1% by weight, less than about 0.05% by weight, or less than about 0.01% by weight of the stated material, based on the total weight of the composition.

As used herein, the terms “substantially identical reference composition,” “substantially identical reference article,” or “substantially identical reference electrochemical cell” refer to a reference composition, article, or electrochemical cell comprising substantially identical components in the absence of an inventive component. In another exemplary aspect, the term “substantially,” in, for example, the context “substantially identical reference composition,” or “substantially identical reference article,” or “substantially identical reference electrochemical cell” refers to a reference composition, article, or an electrochemical cell comprising substantially identical components and wherein an inventive component is substituted with a common in the art component.

The devices, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods, in addition to those shown and described herein, are intended to fall within the scope of the appended claims. Further, while only certain representative devices, systems, and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less; however, other combinations of steps, elements, components, and constituents are included, even though not explicitly stated.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove and that many modifications and other embodiments are intended to be included within the scope of the appended claims. Moreover, although specific terms are employed herein, as well as in the claims which follow, they are used only in a generic and descriptive sense and not for the purposes of limiting the described invention nor the claims which follow.

Unless defined otherwise, all technical and scientific terms used herein have the same meanings as commonly understood by one of skill in the art to which the disclosed invention belongs. Publications cited herein and the materials for which they are cited are specifically incorporated by reference.

While aspects can be described and claimed in a particular statutory class, such as the system statutory class, this is for convenience only and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

In view of the described processes and compositions, hereinbelow are described certain more particularly described aspects of the inventions. These particularly recited aspects should not, however, be interpreted to have any limiting effect on any different claims containing different or more general teachings described herein, or that the “particular” aspects are somehow limited in some way other than the inherent meanings of the language and formulas literally used therein.

The present invention may be understood more readily by reference to the following detailed description of various aspects of the invention and the examples included therein and to the Figures and their previous and following description.

What is claimed is:

1. A method of reducing or preventing central venous stenosis in a patient having a percutaneously inserted central venous catheter present within a vein, the method comprising:
providing central venous catheter comprising:
a catheter body having an exterior surface and comprising a proximal end, a distal end, and a mid-section; and
a main lumen having an interior lumen wall, the main lumen running along a length of said catheter, thereby defining a main axis, and terminating at a distal lumen orifice, wherein such distal lumen orifice is arranged to provide liquid communication from said main lumen to the vein when the central venous catheter is percutaneous inserted within the vein;
disposing a lubricant coating on at least a portion of the exterior surface of the catheter body disposed with in the vein when the central venous catheter is percutaneous inserted within the vein; and
percutaneously inserting the central venous catheter within the vein of the patient; wherein the lubricant coating reduces friction between the exterior surface of the catheter body and an endothelial wall of the vein.

2. The method of claim 1, wherein the lubricant coating exhibits a coefficient of friction of 0.5 or less with the endothelial wall of the vein.

3. The method of claim 1, wherein the lubricant coating comprises a roughened substrate and a lubricating fluid layer immiscible with a biological material, the lubricating fluid layer adhering to and preferentially wetting the roughened substrate to form a slippery surface over the roughened substrate.

4. The method of claim 3, wherein the roughened substrate comprises a polymer.

5. The method of claim 4, wherein the roughened substrate comprises a porous polymer.

6. The method of claim 5, wherein the porous polymer comprises PTFE, ePTFE, silicon, polyurethane, fluorinated ethylene propylene, or combinations thereof.

7. The method of claim 3, wherein the roughened substrate comprises roughened features of 50 nm to 1 mm.

8. The method of claim 3, wherein the lubricating fluid comprises a fluorocarbon.

9. The method of claim 8, wherein the fluorocarbon comprises perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1HPerfluoropentane, HFA 134aTM, HFA227eaTM, methyl perfluorobutylether, methyl perfluoropropyl ether (3M Novec 7000™), 2,2,2-trifluoroethanol, silicone solutions, and combinations thereof.

10. The method of claim 3, wherein the lubricating fluid further comprises carbohydrates, proteins, fats, vitamins, minerals, electrolytes, and combinations thereof.

11. The method of claim 3, wherein the lubricating fluid is insoluble and immiscible in water.

12. The method of claim 1, wherein the catheter interior lumen wall further comprises an anti-biofouling coating.

13. The method of claim 12, wherein the anti-biofouling coating comprises a roughened substrate and a lubricating fluid layer immiscible with a biological material, the lubricating fluid layer adhering to and preferentially wetting the roughened substrate to form a slippery surface over the roughened substrate.

14. The method of claim 13, wherein the roughened substrate comprises a polymer.

15. The method of claim 14, wherein the roughened substrate comprises a porous polymer.

16. The method of claim 15, wherein the porous polymer comprises PTFE, ePTFE, silicon, polyurethane, or combinations thereof.

17. The method of claim 13, wherein the roughened substrate comprises roughened features of 50 nm to 1 mm.

18. The method of claim 13, wherein the lubricating fluid comprises a fluorocarbon, such as a perfluorocarbon.

19. The method of claim 18, wherein the fluorocarbon comprises perfluoropropane, perfluorobutane, perfluoropentane, perfluorohexane, perfluorooctane, perfluorodecalin, perfluoroperhydrophenanthrene, perfluorooctylbromide, perfluoro tributyl amine, perfluorotripentyl amine, poly(hexafluoropropylene oxide), 1H,4H-perfluorobutane, 1HPerfluoropentane, HFA 134aTM, HFA227eaTM, methyl perfluorobutylether, methyl perfluoropropyl ether (3M Novec 7000™), 2,2,2-trifluoroethanol, silicone solutions, and combinations thereof.

20. The method of claim 13, wherein the lubricating fluid further comprises carbohydrates, proteins, fats, vitamins, minerals, electrolytes, and combinations thereof.
